(19) United States
(12) Patent Application Publication
(10) Pub. No.: US 2006/0142389 A1
(43) Pub. Date: Jun. 29, 2006

(54) Pharmaceutically useful salts of carboxylic acid derivatives

(75) Inventors: Carl-Johan Aurell, Sodertalje (SE); Mikael Dahlstrom, Molndal (SE); Eva-Lotte Lindstedt-Alstermark, Molndal (SE); Anna Minidis, Sodertalje (SE); Bengt Ohlsson, Molndal (SE); Erica Stahle, Sodertalje (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/560,657

(22) PCT Filed: Jun. 16, 2004
(86) PCT No.: PCT/SE04/00964

§ 371 Date: Dec. 13, 2005

(30) Foreign Application Priority Data
Jun. 18, 2003 (GB) 0314129.8

(51) Int. Cl. A61K 31/205; C07C 229/34

(52) U.S. Cl. 514/554; 562/444

(57) ABSTRACT

A compound selected from one or more of the following: a (1R,2S)-2-hydroxyindan-1-amine salt of (2S)-2-ethoxy-3-(4-{2-[hexyl(2-phenylethyl)amino]-2-oxoethoxy}phenyl)propanoic acid; an L-arginine salt of (2S)-2-ethoxy-3-(4-{2-[hexyl(2-phenylethyl)amino-2-oxoethoxyphenyl)propanoic acid; a tert-butylamine salt of (2S)-2-ethoxy-3-(4-{2-[hexyl(2-phenylethyl)amino-2-oxoethoxyphenyl)propanoic acid; a choline salt of (2S)-2-ethoxy-3-(4-{2-[hexyl(2-phenylethyl)amino]-2-oxoethoxy}phenyl)propanoic acid; an adamantylamine salt of (2S)-2-ethoxy-3-(4-{2-[hexyl(2-phenylethyl)amino]-2-oxoethoxy}phenyl)propanoic acid; a N-benzyl-2-phenylethanaminium salt of (2S)-2-ethoxy-3-(4-{2-[hexyl(2-phenylethyl)amino]-2-oxoethoxy}phenyl)propanoic acid; a N-benzyl-2-(benzylamino) ethanaminium salt of (2S)-2-ethoxy-3-(4-{2-[hexyl(2-phenylethyl)amino]-2-oxoethoxy}phenyl)propanoic acid; or a tris(hydroxymethyl)methylamine salt of (2S)-2-ethoxy-3-(4-{2-[hexyl(2-phenylethyl)amino]-2-oxoethoxy}phenyl)propanoic acid.

### FIELD OF THE INVENTION

The present invention relates to certain novel salts of (2S)-3-(4-{2-[amino]-2-oxoethoxy}phenyl)-2-ethoxypropanoic acid derivatives, to processes for preparing such compounds, to their utility in treating clinical conditions including lipid disorders (dyslipidemias) whether or not associated with insulin resistance and other manifestations of the metabolic syndrome, to methods for their therapeutic use and to pharmaceutical compositions containing them.

### BACKGROUND OF THE INVENTION

The metabolic syndrome including type 2 diabetes mellitus, refers to a cluster of manifestations including insulin resistance with accompanying hyperinsulinaemia, possibly type 2 diabetes mellitus, arterial hypertension, central (visceral) obesity, dyslipidaemia observed as deranged lipoprotein levels typically characterised by elevated VLDL (very low density lipoproteins), small dense LDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in patients with the metabolic syndrome and thus to correct the dyslipidaemia which is considered to cause the accelerated progress of atherosclerosis. However, currently this is not a universally accepted diagnosis with well-defined pharmacotherapeutic indications.

Co-pending PCT application No. PCT/GB02/05743 discloses compounds of formula A 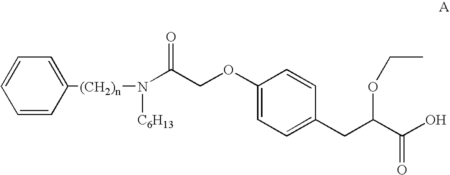
wherein n is 1 or 2 and optical isomers and racemates thereof, pharmaceutically acceptable salts, solvates, crystalline forms and prodrugs thereof are highly potent PPARα modulators. PPAR is short peroxisome proliferator-activated receptors (for for a review of the PPARs see T. M. Willson et al, J Med Chem 2000, Vol 43, 527). These compounds are effective in treating conditions associated with insulin resistance. Specific pharmaceutically acceptable salts of compounds of the formula A are not disclosed in PCT/GB02/05743. Further, no information is provided in relation to how crystalline forms of compounds of the formula A, and particularly salts thereof, may be prepared. The compound in which n is 2 is prepared as the free acid in this application. However, this compound is a syrup and is thus not suitable for use in pharmaceutical formulations. Therefore there exists a need for a derivative of this compound which has physical and chemical properties suitable for use in pharmaceutical formulations. Attempts were made to produce salts with many different counter-ions. However, most were unsatisfactory for one of the following reasons. A salt could not be formed in the solid state or if formed the salt was amorphous with a low glass transition temperature.

In the formulation of drug compositions, it is important for the drug substance to be in a form in which it can be conveniently handled and processed. This is of importance, not only from the point of view of obtaining a commercially-viable manufacturing process, but also from the point of view of subsequent manufacture of pharmaceutical formulations comprising the active compound.

Further, in the manufacture of drug compositions, it is important that a reliable, reproducible and constant plasma concentration profile of drug is provided following administration to a patient.

Chemical stability, solid state stability, and “shelf life” of the active ingredients are also very important factors. The drug substance, and compositions containing it, should preferably be capable of being effectively stored over appreciable periods of time, without exhibiting a significant change in the active component's physico-chemical characteristics (e.g. its chemical composition, density, hygroscopicity and solubility).

Moreover, it is also important to be able to provide drug in a form which is as chemically pure as possible.

The skilled person will appreciate that, typically, if a drug can be readily obtained in a stable form, such as a stable crystalline form, advantages may be provided, in terms of ease of handling, ease of preparation of suitable pharmaceutical formulations, and a more reliable solubility profile.

### DESCRIPTION OF THE INVENTION

The present invention provides a compound selected from one or more of the following:
a (1R,2S)-2-hydroxyindan-1-amine salt of (2S)-2-ethoxy-3-(4-{2-[hexyl(2-phenylethyl)amino]-2-oxoethoxy}phenyl)propanoic acid;
an L-arginine salt of (2S)-2-ethoxy-3-(4-{2-[hexyl(2-phenylethyl)amino-2-oxoethoxyphenyl)propanoic acid;
a tert-butylamine salt of (2S)-2-ethoxy-3-(4-{2-[hexyl(2-phenylethyl)amino-2-oxoethoxyphenyl)propanoic acid;
a choline salt of (2S)-2-ethoxy-3-(4-{2-[hexyl(2-phenylethyl)amino]-2-oxoethoxy}phenyl)propanoic acid;
an adamantylamine salt of (2S)-2-ethoxy-3-(4-{2-[hexyl(2-phenylethyl)amino]-2-oxoethoxy}phenyl)propanoic acid;
a N-benzyl-2-phenylethanaminium salt of (2S)-2-ethoxy-3-(4-{2-[hexyl(2-phenylethyl)amino]-2-oxoethoxy}phenyl)propanoic acid;
a N-benzyl-2-(benzylamino) ethanaminium salt of (2S)-2-ethoxy-3-(4-{2-[hexyl(2-phenylethyl)amino]-2-oxoethoxy}phenyl)propanoic acid; or
   a tris(hydroxymethyl)methylamine salt of (2S)-2-ethoxy-3-(4-{2-[hexyl(2-phenylethyl)amino]-2-oxoethoxy}phenyl)propanoic acid.

We have found that certain compounds of the invention have the advantage that they may be prepared in crystalline form.

According to a further aspect of the invention there is provided a compound of the invention in substantially crystalline form.

Although we have found that it is possible to produce compounds of the invention in forms which are greater than 80% crystalline, by “substantially crystalline” we include greater than 20%, preferably greater than 30%, and more preferably greater than 40% (e.g. greater than any of 50, 60, 70, 80 or 90%) crystalline.

According to a further aspect of the invention there is also provided a compound of the invention in partially crystalline form. By “partially crystalline” we include 5% or between 5% and 20% crystalline.

The degree (%) of crystallinity may be determined by the skilled person using X-ray powder diffraction (XRPD). Other techniques, such as solid state NMR, FT-IR, Raman spectroscopy, differential scanning calorimetry (DSC) and microcalorimetry, may also be used.

Compounds of the invention, and particularly crystalline compounds of the invention, may have improved stability when compared to compounds disclosed in PCT/GB02/05743.

The term “stability” as defined herein includes chemical stability and solid state stability.

By “chemical stability”, we include that it may be possible to store compounds of the invention in an isolated form, or in the form of a formulation in which it is provided in admixture with pharmaceutically acceptable carriers, diluents or adjuvants (e.g. in an oral dosage form, such as a tablet, capsule etc.), under normal storage conditions, with an insignificant degree of chemical degradation or decomposition.

By “solid state stability”, we include that it may be possible to store compounds of the invention in an isolated solid form, or in the form of a solid formulation in which it is provided in admixture with pharmaceutically acceptable carriers, diluents or adjuvants (e.g. in an oral dosage form, such as a tablet, capsule etc.), under normal storage conditions, with an insignificant degree of solid state transformation (e.g. crystallisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, such as 15 to 30° C.), pressures of between 0.1 and 2 bars (preferably at atmospheric pressure), relative humidities of between 5 and 95% (preferably 10 to 60%), and/or exposure to 460 lux of UV/visible light, for prolonged periods (i.e. greater than or equal to six months). Under such conditions, compounds of the invention may be found to be less than 15%, more preferably less than 10%, and especially less than 5%, chemically-degraded/decomposed, or solid state transformed, as appropriate. The skilled person will appreciate that the above-mentioned upper and lower limits for temperature, pressure and relative humidity represent extremes of normal storage conditions, and that certain combinations of these extremes will not be experienced during normal storage (e.g. a temperature of 50° C. and a pressure of 0.1 bar).

It may be possible to crystallise salts of compounds of formula A with or without the presence of a solvent system (e.g. crystallisation may be from a melt, under supercritical conditions, or achieved by sublimation). However, we prefer that crystallisation occurs from an appropriate solvent system.

According to a further aspect of the invention, there is provided a process for the preparation of a crystalline compound of the invention which comprises crystallising a compound of the invention from an appropriate solvent system.

Crystallisation temperatures and crystallisation times depend upon the salt that is to be crystallised, the concentration of that salt in solution, and the solvent system which is used.

Crystallisation may also be initiated and/or effected by way of standard techniques, for example with or without seeding with crystals of the appropriate crystalline compound of the invention.

Different crystalline forms of the compounds of the invention may be readily characterised using X-ray powder diffraction (XRPD) methods, for example as described hereinafter.

In order to ensure that a particular crystalline form is prepared in the absence of other crystalline forms, crystallisations are preferably carried out by seeding with nuclei and/or seed crystals of the desired crystalline form in substantially complete absence of nuclei and/or seed crystals of other crystalline forms. Seed crystals of appropriate compound may be prepared, for example, by way of slow evaporation of solvent from a portion of solution of appropriate salt.

Compounds of the invention may be isolated using techniques which are well known to those skilled in the art, for example decanting, filtering or centrifuging.

Compounds may be dried using standard techniques.

Further purification of compounds of the invention may be effected using techniques, which are well known to those skilled in the art. For example impurities may be removed by way of recrystallisation from an appropriate solvent system. Suitable temperatures and times for the recrystallisation depend upon the concentration of the salt in solution, and upon the solvent system which is used.

When compounds of the invention are crystallised, or recrystallised, as described herein, the resultant salt may be in a form which has improved chemical and/or solid state stability, as mentioned hereinbefore.

Compounds of the invention have the advantage that they may be more efficacious, be less toxic, be longer acting, have a broader range of activity, be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art. Compounds of the invention may have the further advantage that they may be administered less frequently than compounds known in the prior art.

Compounds of the invention may also have the advantage that they are in a form which provides for improved ease of handling. Further, compounds of the invention have the advantage that they may be produced in forms which may have improved chemical and/or solid state stability (including e.g. due to lower hygroscopicity). Thus, such compounds of the invention may be stable when stored over prolonged periods.

Compounds of the invention may also have the advantage that they may be crystallised in good yields, in a high purity, rapidly, conveniently, and at a low cost.

The compounds of the present invention have activity as medicaments. In particular the compounds are highly potent agonists of PPARα. In addition the compounds are also agonists of PPARγ. The term agonists as used herein, includes partial agonists.

It will also be understood that certain crystalline compounds of the present invention may exist in solvated, for example hydrated, as well as unsolvated forms. It is to be understood that the present invention encompasses all such solvated and unsolvated forms.

The present invention also provides the following embodiments.

A (1R,2S)-2-hydroxyindan-1-amine salt of (2S)-2-ethoxy-3-(4-{2-[hexyl(2-phenylethyl)amino]-2-oxoethoxy}phenyl)propanoic acid characterised by an X-ray powder diffraction pattern characterised by peaks with d-values at 20.0, 11.0, 6.5, 4.41, 4.04 and 3.90 Å.

A (1R,2S)-2-hydroxyindan-1-amine salt of (2S)-2-ethoxy-3-(4-{2-[hexyl(2-phenylethyl)amino]-2-oxoethoxy}phenyl)propanoic acid having the XRPD pattern substantially as disclosed in figure A.

A L-arginine salt of (2S)-2-ethoxy-3-(4-{2-[hexyl(2-phenylethyl)amino-2-oxoethoxyphenyl)propanoic acid having the XRPD pattern substantially as disclosed in figure B.

A tert-butylamine salt of (2S)-2-ethoxy-3-(4-{2-[hexyl(2-phenylethyl)amino-2-oxoethoxyphenyl)propanoic acid characterised by an X-ray powder diffraction pattern characterised by peaks with d-values at 18.7, 11.5, 5.9, 5.5, 4.71 and 4.08 Å.

A tert-butylamine salt of (2S)-2-ethoxy-3-(4-{2-[hexyl(2-phenylethyl)amino-2-oxoethoxyphenyl)propanoic acid having the XRPD pattern substantially as disclosed in figure C.

An adamantylamine salt of (2S)-2-ethoxy-3-(4-{2-[hexyl(2-phenylethyl)amino]-2-oxoethoxy}phenyl)propanoic acid.

A N-benzyl-2-phenylethanaminium salt of (2S)-2-ethoxy-3-(4-{2-[hexyl(2-phenylethyl)amino]-2-oxoethoxy}phenyl)propanoic acid acid having the XRPD pattern substantially as disclosed in figure D.

A N-benzyl-2-(benzylamino) ethanaminium salt of (2S)-2-ethoxy-3-(4-{2-[hexyl(2-phenylethyl)amino]-2-oxoethoxy}phenyl)propanoic acid acid having the XRPD pattern substantially as disclosed in figure E.

Methods of Preparation

The compounds of the invention are prepared by dissolving (2S)-2-ethoxy-3-(4-{2-[hexyl(2-phenylethyl)amino]-2-oxoethoxy}phenyl)propanoic acid in an inert solvent at a temperature in the range of 0-100° C. and then adding the appropriate amine either neat or as a solution in an inert solvent and isolating the solid salt. The salt may be isolated by cooling the reaction solution and optionally seeding the solution with the desired product and/or concentrating the solution. Optionally the product may be isolated by adding an antisolvent to a solution of the product in an inert solvent. The solid may be collected by methods known to those skilled in the art for example filtration or centrifugation.

In another aspect the present invention provides the compound obtainable by reacting (2S)-2-ethoxy-3-(4-{2-[hexyl(2-phenylethyl)amino]-2-oxoethoxy}phenyl)propanoic acid and tert-butylamine in an inert solvent, particularly acetone and isolating the product. Particularly an equivalent of tert-butylamine is used.

In another aspect the present invention provides the compound obtainable by reacting (2S)-2-ethoxy-3-(4-{2-[hexyl(2-phenylethyl)amino]-2-oxoethoxy}phenyl)propanoic acid and (1R,2S)-2-hydroxyindan-1-amine in an inert solvent, particularly ethyl acetate and isolating the product. Particularly an equivalent of (1R,2S)-2-hydroxyindan-1-amine is used.

In another aspect the present invention provides the compound obtainable by reacting (2S)-2-ethoxy-3-(4-{2-[hexyl(2-phenylethyl)amino]-2-oxoethoxy}phenyl)propanoic acid and (1R,2S)-2-hydroxyindan-1-amine in an inert solvent, particularly ethyl acetate or isopropyl acetate, and isolating the product. Particularly an equivalent of (1R,2S)-2-hydroxyindan-1-amine is used.

In another aspect the present invention provides the compound obtainable by reacting (2S)-2-ethoxy-3-(4-{2-[hexyl(2-phenylethyl)amino]-2-oxoethoxy}phenyl)propanoic acid and L-arginine in an inert solvent, particularly ethanol or propan-2-ol and isolating the product. Particularly an equivalent of L-arginine is used.

In another aspect the present invention provides the compound obtainable by reacting (2S)-2-ethoxy-3-(4-{2-[hexyl(2-phenylethyl)amino]-2-oxoethoxy}phenyl)propanoic acid and choline in an inert solvent and isolating the product. Particularly an equivalent of choline is used.

In another aspect the present invention provides the compound obtainable by reacting (2S)-2-ethoxy-3-(4-{2-[hexyl(2-phenylethyl)amino]-2-oxoethoxy}phenyl)propanoic acid and tris(hydroxymethyl)methylamine and isolating the product. Particularly an equivalent of tris(hydroxymethyl)methylamine is used.

The expression “inert solvent” refers to a solvent that does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations in a pharmaceutically acceptable dosage form. Depending upon the disorder and patient to be treated and the route of administration, the compositions may be administered at varying doses.

Suitable daily doses of the compound of the invention in therapeutical treatment of humans are about 0.0001-100 mg/kg body weight, preferably 0.001-10 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is thus provided a pharmaceutical formulation including the compound of the invention in admixture with pharmaceutically acceptable adjuvants, diluents and/or carriers.

Pharmacological Properties

The compounds of the invention are useful for the prophylaxis and/or treatment of clinical conditions associated with inherent or induced reduced sensitivity to insulin (insulin resistance) and associated metabolic disorders (also known as metabolic syndrome). These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoAI particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manifestations of the metabolic syndrome. Treatment with the present compounds is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of its insulin sensitizing effect the compound is also expected to prevent or delay the development of type 2 diabetes from the metabolic syndrome and diabetes of pregnancy. Therefore the development of long-term complications associated with chronic hyperglycaemia in diabetes mellitus such as the micro-angiopathies causing renal disease, retinal damage and peripheral vascular disease of the lower limbs are expected to be delayed. Furthermore the compound may be useful in treatment of various conditions outside the cardiovascular system whether or not associated with insulin resistance, like polycystic ovarian syndrome, obesity, cancer and states of inflammatory disease including neurodegenerative disorders such as mild cognitive impairment, Alzheimer's disease, Parkinson's disease and multiple sclerosis.

The compounds of the present invention are expected to be useful in controlling glucose levels in patients suffering from type 2 diabetes.

The present invention provides a method of treating or preventing dyslipidemias, the insulin resistance syndrome and/or metabolic disorders (as defined above) comprising the administration of a compound of the present invention to a mammal (particularly a human) in need thereof.

The present invention provides a method of treating or preventing type 2 diabetes comprising the administration of an effective amount of a compound of the present invention to a mammal (particularly a human) in need thereof.

In a further aspect the present invention provides the use of a compound of the present invention as a medicament.

In a further aspect the present invention provides the use of a compound of the present invention in the manufacture of a medicament for the treatment of insulin resistance and/or metabolic disorders.

Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. The compound of the invention may be combined with another therapeutic agent that decreases the ratio of LDL:HDL or an agent that causes a decrease in circulating levels of LDL-cholesterol. In patients with diabetes mellitus the compound of the invention may also be combined with therapeutic agents used to treat complications related to micro-angiopathies.

A compound of the invention may be used alongside other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or nateglinide.

In another aspect of the invention, the compound of formula I, or a pharmaceutically acceptable salt thereof, may be administered in association with a PPAR modulating agent. PPAR modulating agents include but are not limited to a PPAR alpha and/or gamma and/or delta agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 04/000790, WO 04/000295, WO 04/000294, WO 03/051822, WO 03/051821, WO 02/096863, WO 03/051826, WO 02/085844, WO 01/040172, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, NN-2344), clofibrate, fenofibrate, bezafibrate, gemfibrozil, ciprofibrate, pioglitazone, rosiglitazone, AVE-0847, AVE-8134, CLX-0921, DRF-10945, DRF-4832, LY-518674, LY-818, LY-929, 641597, GW-590735, GW-677954, GW-501516, MBX-102, ONO-5129, KRP-101, R483 (BMM131258), TAK-559 or TAK-654. Particularly a PPAR alpha and/or gamma and/or delta agonist refers to tesaglitazar ((S)-2-ethoxy-3-[4-(2-{4-methanesulphonyl-oxyphenyl}ethoxy)phenyl]propanoic acid) and pharmaceutically acceptable salts thereof.

In addition a compound of the invention may be used in conjunction with a sulfonylurea for example: glimepiride, glibenclamide (glyburide), gliclazide, glipizide, gliquidone, chloropropaamide, tolbutamide, acetohexamide, glycopyramide, carbutamide, glibonuride, glisoxepid, glybuthiazole, glibuzole, glyhexamide, glymidine, glypinamide, phenbutamide, tolcylamide and tolazamide. Preferably the sulfonylurea is glimepiride or glibenclamide (glyburide). More preferably the sulfonylurea is glimepiride. The present invention includes administration of a compound of the present invention in conjunction with one, two or more existing therapies described in this combination section. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin selected from the group consisting of atorvastatin, bervastatin, cerivastatin, dalvastatin, fluvastatin, itavastatin, lovastatin, mevastatin, nicostatin, nivastatin, pravastatin and simvastatin, or a pharmaceutically acceptable salt, especially sodium or calcium, or a solvate thereof, or a solvate of such a salt. A particular statin is atorvastatin, or a pharmaceutically acceptable salt, solvate, solvate of such a salt or a prodrug thereof. A more particular statin is atorvastatin calcium salt. A particularly preferred statin is, however, a compound with the chemical name (E)-7-[4-(4-fluorophenyl)-6-isopropyl-2-[methyl(methylsulfonyl)-amino]-pyrimidin-5-yl](3R,5S)-3,5-dihydroxyhept-6-enoic acid, [also known as (E)-7-[4-(4-fluorophenyl)-6-isopropyl-2-[N-methyl-N-(methylsulfonyl)-amino]pyrimidin-5-yl](3R,5S)-3,5-dihydroxyhept-6-enoic acid]or a pharmaceutically acceptable salt or solvate thereof, or a solvate of such a salt. The compound (E)-7-[4-(4-fluorophenyl)-6-isopropyl-2-[methyl-(methylsulfonyl)-amino]-pyrimidin-5-yl](3R,5S)-3,5-dihydroxyhept-6-enoic acid, and its calcium and sodium salts are disclosed in European Patent Application, Publication No. EP-A-0521471, and in Bioorganic and Medicinal Chemistry, (1997), 5(2), 437-444. This latter statin is now known under its generic name rosuvastatin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with a bile acid sequestering agent, for example colestipol or cholestyramine or cholestagel.

The present invention also includes a compound of the present invention in combination with an inhibitor of the ileal bile acid transport system (IBAT inhibitor).

Suitable compounds possessing IBAT inhibitory activity have been described, see for instance the compounds described in WO 93/16055, WO 94/18183, WO 94/18184, WO 96/05188, WO 96/08484, WO 96/16051, WO 97/33882, WO 98/07449, WO 98/03818, WO 98/38182, WO 99/32478, WO 99/35135, WO 98/40375, WO 99/35153, WO 99/64409, WO 99/64410, WO 00/01687, WO 00/47568, WO 00/61568, WO 00/62810, WO 01/68906, DE 19825804, WO 00/38725, WO 00/38726, WO 00/38727, WO 00/38728, WO 00/38729, WO 01/68906, WO 01/66533, WO 02/32428, WO 02/50051, EP 864 582, EP489423, EP549967, EP573848, EP624593, EP624594, EP624595 and EP624596 and the contents of these patent applications are incorporated herein by reference. Further suitable compunds possessing IBAT inhibitory activity have been described in WO 94/24087, WO 98/56757, WO 00/20392, WO 00/20393, WO 00/20410, WO 00/20437, WO 01/34570, WO 00/35889, WO 01/68637, WO 02/08211, WO 03/020710, WO 03/022825, WO 03/022830, WO 03/022286, WO 03/091232, WO 03/106482, JP 10072371, U.S. Pat. No. 5,070,103, EP 251 315, EP 417 725, EP 869 121, EP 1 070 703 and EP 597 107 and the contents of these patent applications are incorporated herein by reference.

Particular classes of IBAT inhibitors suitable for use in the present invention are benzothiepines, and the compounds described in the claims, particularly claim 1, of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity is (3R,5R)-3-butyl-3-ethyl-1,1-dioxido-5-phenyl-2,3,4,5-tetrahydro-1,4-benzothiazepin-8-yl □-D-glucopyranosiduronic acid (EP 864 582). Other suitable IBAT inhibitors include one of:
1,1-dioxo-3,3-dibutyl-5-phenyl-7-methylthio-8-(N-{(R)-1′-phenyl-1′-[N-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4 -hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)cabamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)-N′-(2-methylsulphinyl-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
1,1-dioxo-3,3-dibutyl-5-phenyl-7-methylthio-8-(N-{(R)-α-[N-((R)-1-carboxy-2-methylthio-ethyl)carbamoyl]4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl)carbamoyl]4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N-((S)-2-hydroxy-1-carboxyethyl)carbamnoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]benzyl}carbamoylmethoxy)-2,3,4,5-tetrahydro-1,2,5-benzothiadiazepine;
1,1-Dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N-((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbaimoyl]-4-hydroxybenzyl}carbamoylmethoxy)-2,3,4,5-tetrahydro-1,2,5-benzothiadiazepine; and
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof.

According to an additional further aspect of the present invention there is provided a combination treatment comprising the administration of an effective amount of a compound of the present invention the formula A optionally together with a pharmaceutically acceptable diluent or carrier, with the simultaneous, sequential or separate administration one or more of the following agents selected from:

a CETP (cholesteryl ester transfer protein) inhibitor, for example those referenced and described in WO 00/38725 page 7 line 22-page 10, line 17 which are incorporated herein by reference;

a cholesterol absorption antagonist for example azetidinones such as SCH 58235 and those described in U.S. Pat. No. 5,767,115 which are incorporated herein by reference;

a MTP (microsomal transfer protein) inhibitor for example those described in Science, 282, 751-54, 1998 which are incorporated herein by reference;

a nicotinic acid derivative, including slow release and combination products, for example, nicotinic acid (niacin), acipimox and niceritrol;

a phytosterol compound for example stanols;

probucol;

an omega-3 fatty acid for example Omacor™;

an anti-obesity compound for example orlistat (EP 129,748) and sibutramine (GB 2,184,122 and U.S. Pat. No. 4,929,629);

an antihypertensive compound for example an angiotensin converting enzyme (ACE) inhibitor, an angiotensin II receptor antagonist, an andrenergic blocker, an alpha andrenergic blocker, a beta andrenergic blocker for example metoprolol, a mixed alpha/beta andrenergic blocker, an andrenergic stimulant, calcium channel blocker, an AT-1 blocker, a saluretic, a diuretic or a vasodilator;

a CB 1 antagonist or inverse agonist for example as described in W001/70700 and EP 65635; aspirin;

a Melanin concentrating hormone (MCH) antagonist;

a PDK inhibitor; or

modulators of nuclear receptors for example LXR, FXR, RXR, and RORalpha;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Particular ACE inhibitors or pharmaceutically acceptable salts, solvates, solvate of such salts or a prodrugs thereof, including active metabolites, which can be used in combination with a compound of the invention include but are not limited to, the following compounds: alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril; spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Preferred ACE inhibitors for use in the present invention are ramipril, ramiprilat, lisinopril, enalapril and enalaprilat. More preferred ACE inhibitors for uses in the present invention are ramipril and ramiprilat.

Preferred angiotensin II antagonists, pharmaceutically acceptable salts, solvates, solvate of such salts or a prodrugs thereof for use in combination with a compound of the invention include, but are not limited to, compounds: candesartan, candesartan cilexetil, losartan, valsartan, irbesartan, tasosartan, telmisartan and eprosartan. Particularly preferred angiotensin II antagonists or pharmaceutically acceptable derivatives thereof for use in the present invention are candesartan and candesartan cilexetil.

Therefore in an additional feature of the invention, there is provided a method for for the treatment of type 2 diabetes and its associated complications in a warm-blooded animal, such as man, in need of such treatment which comprises administering to said animal an effective amount of a compound of the present invention in simultaneous, sequential or separate administration with an effective amount of one the other compounds described in this combination section, or a pharmaceutically acceptable salt, solvate, solvate of such a salt or a prodrug thereof.

Therefore in an additional feature of the invention, there is provided a method of treating hyperlipidemnic conditions in a warm-blooded animal, such as man, in need of such treatment which comprises administering to said animal an effective amount of a compound of the present invention of a compound of the invention in simultaneous, sequential or separate administration with an effective amount of one the other compounds described in this combination section or a pharmaceutically acceptable salt, solvate, solvate of such a salt or a prodrug thereof.

According to a further aspect of the invention there is provided a pharmaceutical composition which comprises a compound of the present invention and one of the other compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of the present invention and one of the other compounds described in this combination section or a pharmaceutically acceptable salt, solvate, solvate of such a salt or a prodrug thereof.

According to a further aspect of the present invention there is provided a kit comprising:

a) a compound of the present invention in a first unit dosage form;

b) one of the other compounds described in this combination section or a pharmaceutically acceptable salt, solvate, solvate of such a salt or a prodrug thereof; in a second unit dosage form; and

c) container means for containing said first and second dosage forms.

According to a further aspect of the present invention there is provided a kit comprising:

a) a compound of the present invention together with a pharmaceutically acceptable diluent or carrier, in a first unit dosage form;

b) one of the other compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the present invention of the present invention and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the present invention and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the present invention optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Experimental

¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard.

X-ray powder diffraction analysis (XRPD) was performed using variable slits on samples prepared according to standard methods with and/or without using an internal standard. Standard methods are, for example described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using a Siemens D5000 diffractometer or a Philips X'Pert MPD. X-ray analyses were performed using Cu-radiation a Siemens D5000 diffractometer and a Philips X'Pert PD. The X-axis in the figures below is 2-theta and the Y axis is intensity.

Differential scanning calorimetry (DSC) was performed using a Mettler DSC820, a Mettler DSC820E or a Perkin Elmer DSC 7 instrument, according to standard methods, for example hose described in Hbhne, G. W. H. et al (1996), Differential Scanning Calorimetry, Springer, Berlin.

Thermogravimetric analysis (TGA) was performed using a Mettler Toledo TGA850, a Mettler Toledo TG851 or a Perkin Elmer TGA 7 instrument.

It will be appreciated by the skilled person that crystalline forms of compounds of the invention may be prepared by analogy with processes described herein and/or in accordance with the Examples below, and may show essentially the same XRPD diffraction patterns and/or DSC and/or TGA thermograms as those disclosed herein. By “essentially the same” XRPD diffraction patterns and/or DSC and/or TGA thermograms, we include those instances when it is clear from the relevant patterns and/or thermograms (allowing for experimental error) that essentially the same crystalline form has been formed. When provided, DSC onset temperatures may vary in the range ±5° C. (e.g. ±2° C.), and XRPD distance values may vary in the range ±2 on the last decimal place. It will be appreciated by the skilled person that XRPD intensities may vary when measured for essentially the same crystalline form for a variety of reasons including, for example, preferred orientation.

Abbreviations

NMR Abbreviations
t triplet
s singlet
d doublet
q quartet
m multiplet
bs broad singlet

XRPD Abbreviations
XRPD X-ray powder diffraction

d-value the spacing between successive parallel hkl planes in a crystal lattice

| Intensity (rel %) | Definition |
|---|---|
| 25-100 | vs (very strong) |
| 10-25 | s (strong) |
| 3-10 | m (medium) |
| 1-3 | w (weak) |

TGA thermogravimetric analysis
DSC differential scanning calorimetry

### EXAMPLES

Preparation of Starting Material

Method 1

### (2S)-2-Ethoxy-3-(4-{2-[hexyl(2-phenylethyl)amino]-2-oxoethoxyl}phenyl)propanoic acid

### (i) Ethyl (2S)-3-{4-[2-(benzyloxy)-2-oxoethoxy]phenyl}-2-ethoxypropanoate

To a solution of ethyl (2S)-2-ethoxy-3-(4-hydroxyphenyl)propanoate (23.8 g, 100 mmol, prepared as described in WO99/62872) in acetonitrile (200 mL) was added anhydrous potassium carbonate (31.9 g, 231 mmol) followed by benzyl bromoacetate (17.4 mL, 110 mmol) and the reaction mixture was refluxed overnight. The reaction mixture was allowed to cool to room temperature, insoluble salts were filtered off and the solution was concentrated in vacuo. The residue was taken up in ethyl acetate (300 mL), and the organic phase was washed with aqueous NaHCO₃ (3×100 mL) and brine (100 mL), dried over anhydrous MgSO₄, and concentrated in vacuo. Purification on silica gel with methylene chloride as the eluent and collection of pure fractions yielded 22.4 g (58%) of a yellow oil.

¹H NMR (400 MHz, CDCl₃): δ 1.16 (t, 3H), 1.22 (t, 3H), 2.93-2.97 (m, 2H), 3.35 (m, 1H), 3.60 (m, 1H), 3.97 (m, 1H), 4.16 (q, 2H), 4.64 (s, 2H), 5.23 (s, 2H), 6.82 (d, 2H), 7.15 (d, 2H), 7.32-7.39 (m, 5H).

¹³C NMR (100 MHz, CDCl₃): δ 14.3, 15.2, 38.6, 60.9, 65.6, 66.3, 67.0, 80.4, 114.6, 128.5, 128.6, 128.7, 130.6, 135.3, 156.7, 169.0, 172.6.

### (ii) {4-[(2S)-2,3-Diethoxy-3-oxopropyl]phenoxy}acetic acid

To a solution of ethyl (2S)-3-{4-[2-(benzyloxy)-2-oxoethoxy]phenyl}-2-ethoxypropanoate (22.33 g, 57.8 mmol) in freshly distilled THF (290 mL) was added Pd/C (10%, 3.1 g) and the reaction mixture was hydrogenated under atmospheric pressure at room temperature overnight.

The mixture was filtered through a plug of Celite and the filtrate was concentrated in vacuo to afford 16.6 g (97%) of a light yellow oil.

¹H NMR (400 MHz, CDCl₃): δ 1.15 (t, 3H), 1.21 (t, 3H), 2.93-2.98 (m, 2H), 3.35 (m, 1H), 3.60 (m, 1H), 3.97 (m, 1H), 4.16 (q, 2H), 4.65 (s, 2H), 6.84 (d, 2H), 7.17 (d, 2H), 8.48 (bs, 1H)

¹³C NMR (100 MHz, CDCl₃): δ 14.3, 15.1, 38.5, 61.0, 65.1, 66.4, 80.3, 114.6, 130.7, 130.9, 156.4, 172.7, 173.7

### (iii) Ethyl (2S)-2-ethoxy-3-(4-{2-[hexyl(2-phenylethyl)amino]-2-oxoethoxy}phenyl) propanoate

To a solution of {4-[(2S)-2,3-diethoxy-3-oxopropyl]phenoxy}acetic acid (0.110 g, 0.37 mmol) in methylene chloride (3.7 mL) were added N-hexyl-2-phenylethylamine (0.080 g, 0.39 mmol) and DMAP (0.045 g, 0.37 mmol) followed by 1-ethyl-3-(3-dimethylaminopropyl)carbodiimide hydrochloride (0.071 g, 0.37 mmol), and the reaction mixture was stirred at room temperature overnight. The mixture was diluted with methylene chloride (25 mL), and the organic phase was washed with 5% HCl (3×25 mL), aqueous NaHCO₃ (25 mL) and brine (25 mL), dried over Na₂SO₄, and concentrated in vacuo. Purification on a prepacked column of silica gel (Isolute® SPE Column, 5 g Si/25 mL) with methanol (0-1% gradient) in methylene chloride as the eluent yielded 0.125 g (70%) of a colourless oil.

¹H NMR (400 MHz, CDCl₃): δ 0.82-0.92 (m, 3H), 1.16 (t, 3H), 1.19-1.33 (m, 9H), 1.45-1.65 (m, 2H), 2.82-2.90 (m, 2H), 2.91-2.98 (m, 2H), 3.12-3.21 and 3.29-3.42 (2m, 3H, rotamers) 3.50-3.65 (m, 3H), 3.95 (m,1H), 4.16 (q, 2H), 4.39 and 4.65 (2s, 2H, rotamers), 6.75 and 6.86 (2d, 2H, rotamers), 7.10-7.34 (m, 7H).

¹³C NMR (100 MHz, CDCl₃): δ 14.0, 14.1, 14.3, 15.1, 22.6, 26.5, 26.7, 27.4, 29.0, 31.5, 31.6, 33.9, 35.3, 38.5, 45.9, 48.1, 48.3, 48.9, 60.8, 66.2, 67.5, 80.4, 114.5, 126.4, 126.9, 128.5, 128.9, 130.1, 130.2, 130.5, 130.5, 138.3, 139.2, 156.9, 157.0, 167.6, 167.8, 172.5. (The number of peaks is larger than the number of carbon atoms due to rotamers.)

### (iv) (2S)-2-Ethoxy-3-(4-{2-[hexyl(2-phenylethyl)amino]-2-oxoethoxy}phenyl)propanoic acid

To a solution of ethyl (2S)-2-ethoxy-3-(4-{2-[hexyl(2-phenylethyl)amino]-2-oxoethoxy}phenyl)propanoate (0.081 g, 0.17 mmol) in THF (8.6 mL) was added 4.3 mL of a 0.10 M LiOH solution and the reaction mixture was stirred at room temperature overnight. The reaction mixture was acidified with 2M HCl and extracted with ethyl acetate (3×25 mL). The combined organic phase was washed with brine (25 mL), dried over Na₂SO₄, and concentrated in vacuo to afford 0.073 g (96%) of a colourless oil.

¹H NMR (400 MHz, CDCl₃): δ 0.82-0.93 (m, 3H), 1.15 (t, 3H), 1.20-1.35 (m, 6H), 1.47-1.62 (m, 2H), 2.80-2.99 (m, 3H), 3.00-3.09 (m, 1H), 3.11-13.21 and 3.31-3.44 (2m, 3H, rotamers), 3.50-3.67 (m, 3H), 4.01 (m, 1H), 4.40 and 4.66 (2s, 2H, rotamers), 6.75 and 6.85 (2d, 2H, rotamers), 7.10-7.35 (m, 7H), 8.86 (bs, 1H).

¹³C NMR (100 MHz, CDCl₃): δ 14.0, 14.1, 15.1, 22.6, 22.6, 26.6, 26.7, 27.3, 28.9, 31.5, 31.6, 33.8, 35.2, 38.1, 46.1, 48.3, 48.4, 49.0, 66.7, 67.4, 79.9, 114.6, 126.4, 127.0, 128.6, 128.9, 130.0, 130.1, 130.6, 130.7, 138.2, 139.1, 156.9, 157.0, 168.1, 168.2, 175.6. (The number of peaks is larger than the number of carbon atoms due to rotamers.)

Method 2

### (2S)-2-ethoxy-3-(4-{2-[hexyl(2-phenylethyl)amino]-2-oxoethoxy}phenyl)propanoic acid

a) Phenethylamine (30.0 g) was treated with 6M aqueous sodium hydroxide (61.5 ml) in toluene (100 ml). A solution of chloroacetyl chloride (28.0 g) in toluene (50 ml) was added under temperature control. After complete reaction, the reaction slurry was warmed until a complete solution was obtained, and the water-phase was removed. The organic phase was washed with aqueous hydrogen chloride and water. The resulting toluene phase was reduced by evaporation and diisopropylether was added to the toluene solution. The solution was cooled and 1-chloro-N-phenethylacetamide (42.3 g) was collected by filtration, washed and dried. The product was analysed by LC (99.8 area %) and NMR.

¹H NMR δ_{H}(400 MHz, CDCl₃): 2.88 (t, 2H), 3.60 (dd, 2H), 4.05 (s, 2H), 6.62 (bs, 1H), 7.19-7.58 (m, 5H).

b) A mixture of potassium carbonate (31.5 g), 1-chloro-N-phenethylacetamide (15.0 g), ethyl (2S)-2-ethoxy-3-(4-hydroxyphenyl)propanoate (18.1 g) (see WO 99/62871) and acetonitrile (150 ml) was stirred and brought to the boil under reflux. After complete reaction, the mixture wass cooled and the inorganic salts were filtered off and washed with acetonitrile. The remaining solution was reduced by distillation and the product was crystallised from ethyl acetate and hexanes. Ethyl (2S)-2-ethoxy-3-(4-{2-oxo-2-[(2-phenylethyl)amino]ethoxy}phenyl) propanoate (24.5 g) was collected by filtration, washed and dried. The product was analysed by LC (98.6 area %) and NMR.

¹H NMR δ_{H}(400 MHz, CDCl₃): 1.18 (t, 3H), 1.26 (t, 3H), 2.86 (t, 2H), 2.96-3.01 (m, 2H), 3.37 (dq, 1H), 3.58-3.68 (m, 3H), 4.00 (dd, 1H), 4.20 (q, 2H), 4.47 (s, 2H), 6.65 (bs, 1H), 6.79 (dm, 2H), 7.14-7.36 (m, 7H).

c) A solution of ethyl (2S)-2-ethoxy-3-(4-{2-oxo-2-[(2-phenylethyl)amino]ethoxy}-phenyl)propanoate (36.0 g) in THF (270 ml) was added to a solution of lithium hydroxide (6.51 g) dissolved in water (360 ml). The mixture was stirred at room temperature. After complete reaction, the mixture was evaporated under reduced pressure to remove THF. After evaporation, the reaction mixture was cooled to room temperature and acidified with hydrochloric acid. The acidified product was extracted with ethyl acetate. The ethyl acetate solution was washed with water and evaporated to a reduced volume. The product was crystallised from ethyl acetate and diisopropyl ether. (2S)-2-Ethoxy-3-(4-{2-oxo-2-[(2-phenylethyl)amino]ethoxy}phenyl)-propanoic acid (28.0 g) was filtered off and washed with diisopropyl ether and dried under vacuum.

¹H NMR δ_{H}(400 MHz, CDCl₃): 1.20 (t, 3H), 2.85 (t, 2H), 3.00 (dd, 1H), 3.10 (dd, 1H), 3.46 (dq, 1H), 3.56-3.71 (m, 3H), 4.07 (dd, 1H), 4.45 (s, 2H), 6.68 (bs, 1H), 6.78 (dm, 2H), 7.10-7.38 (m, 7H).

d) Dimethylsulfoxide (DMSO) (2750 mL), potassium hydroxide powder (244 g) and (2S)-2-ethoxy-3-(4-{2-oxo-2-[(2-phenylethyl)amino]ethoxy}phenyl)propanoic acid (250 g) were stirred at approximately 18° C. for ca 20 minutes. 1-Bromohexane (344 g=292 mL) was added over 2.5 hours. The reaction mixture was stirred for approximately 10 minutes. Diisopropyl ether (1000 mL) was added followed by filtration, extraction and separation of the mixture. The DMSO layer was further extracted with diisopropyl ether (2×1000 mL). The DMSO layer was acidified with 4M HCl(aq) (950 mL). Diisopropyl ether (3000 mL) and water (2500 mL) were added followed by extraction. The layers were separated (pH˜2 of aq layer) and the diisopropyl ether layer was washed with water (2500 mL). The diisopropyl ether layer was concentrated in vacuo to a clear, very viscous oil. Yield 317 g, assay 88.1%, corrected yield 91.1%, LC-purity 97.2%, e.e. 97.8%. LC-purity and kiral LC in accordance with reference sample.

¹H NMR δ_{H}(400 MHz, CDCl₃): 0.75-0.85 (m, 3H), 1.10 (t, 3H), 1.14-1.29 (m, 6H), 1.40-1.55 (m, 2H), 2.76-2.93 (m, 3H), 2.97-3.06 (m, 1H), 3.06-3.14 and 3.28-3.43 (2m, 3H, rotamers), 3.45-3.58 (m, 3H), 3.98 (m, 1H), 4.32 and 4.59 (2s, 2H, rotamers), 6.68 and 6.80 (2dm, 2H, rotamers), 7.02-7.31 (m, 8H).

### Example 1

### (1R,2S)-2-hydroxyindan-1-amine salt of (2S)-2-ethoxy-3-(4-{2-[hexyl(2-phenylethyl)amino]-2-oxoethoxy}phenyl)propanoic acid

(2S)-2-Ethoxy3-(4-{2-[hexyl(2-phenylethyl)amino]-2-oxoethoxy}phenyl)propanoic acid (1.51 g) was dissolved in ethyl acetate (15 ml/g) at room temperature. Then (1R,2S)-(+)-cis-amino-2-indanol (1 mole equiv) was added to the solution, followed by addition of seed. The slurry was stirred at room temperature, and the product (1.89 g) was filtered off to give the title compound which was confirmed with XRPD and NMR.

¹H-NMR (400 MHz, CDCl₃): 7.5 (1H, d), 7.4-7.1 (10H, m), 6.8 (1H, d), 6.6 (1H, d), 6.4 (4H, br s), 4.6 (2H, m), 4.4 (2H, m), 3.9 (1H, m), 3.5 (3H, m), 3.43.2 (2H, m), 3.2-3.0 (3H, m), 2.9 (4H, m), 1.5 (2H, br m),1.3 (6H, br s), 1.1 (3H, m), 0.9 (3H, m).

### Example 2

### (1R,2S)-2-hydroxyindan-1-amine (2S)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (109 mg) and (1R,2S)-(+)-cis-1-amino-2-indanol (36 mg) were dissolved in ethyl acetate (1.4 ml) and stirred at room temperature. When a salt had precipitated ethyl acetate was added (3.2 ml). The slurry was stirred at room temperature, filtered and the solids washed with ethyl acetate (1 ml) and dried by suction. The product was confirmed as (1R,2S)-2-hydroxyindan-1-aminium (2S)-2-ethoxy-3-(4-{2-[hexyl(2-phenylethyl)amino]-2-oxoethoxy}phenyl)propanoate with LC and XRPD.

### Example 3

### (1R,2S)-2-hydroxyindan-1-amine salt (2S)-2-ethoxy-3-(4-{2-[hexyl(2-phenylethyl)amino]-2-oxoethoxyy}phenyl)propanoic acid

(2S)-2-Ethoxy-3-(4-{2-[hexyl(2-phenylethyl)amino]-2-oxoethoxy}phenyl)propanoic acid (1.00 g) and (1R,2S)-(+)-cis-1-amino-2-indanol (0.27 g) were dissolved in isopropyl acetate (40 ml) and stirred at room temperature. When a salt had precipitated the slurry was filtered and the solids washed with isopropyl acetate (20 ml) and dried by suction to give 0.97 g of title compound.

### Example 4

### L-arginine salt (2S)-2-ethoxy-3-(4-{2-[hexyl(2-phenylethyl)amino-2-oxoethoxyphenyl)propanoic acid

L-Arginine, (11.32 g) was dissolved in 25 ml of distilled water at 60° C. The warm clear solution of L-arginine in water was added under stirring to a solution of (2S)-2-ethoxy3-(4-{2-[hexyl(2-phenylethyl)amino]-2-oxoethoxy}phenyl)propanoic acid (33 g) in 2-propanol (150 ml). The resulting solution was evaporated to an oil which was precipitated by adding 150 ml of isopropyl acetate under stirring. The amorphous salt was filtered off and dried under vacuum at 40° C. The yield was 36 g. A small amount of material was dissolved in warm butyl acetate and was re-precipitated by addition of cyclohexane. This material was dried in vacuum at 40° C. and used for NMR analysis.

¹H-NMR (400 MHz, MeOD): 7.2 (7H, m), 6.9 (1H,d), 6.7 (1H, d), 4.7 (1H, s), 4.4 (1Hs), 3.8 (1H, m), 3.6 (4H, m), 3.4 (1H, t), 3.2 (4H, m), 2.9 (4H, m), 1.9 (2H, m), 1.7 (2H, m), 1.6 (2H, br m), 1.3 (6H, br s), 1.1 (3H, t), 0.9 (3H, t)

### Example 5

### L-arginine salt (2S)-2-ethoxy-3-(4-{2-[hexyl(2-phenylethyl)amino]-2-oxoethoxy}phenyl)propanoic acid

The (2S)-2-ethoxy3-(4-{2-[hexyl(2-phenylethyl)amino]-2-oxoethoxy}phenyl)propanoic acid (0.197 g) was dissolved in 95% ethanol and L-arginine (1 mole equiv) was added to the solution. The solution was stirred at ambient temperature, followed by evaporation to dryness and addition of isooctane (10 ml/g). The slurry was stirred at room temperature, and the product was filtered off and analysed by XRPD.

### Example 6

### tert-butylamine salt of (2S)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (0.49 g) and tert-butylamine (0.077 g) were mixed in acetone(8 ml/g), followed by addition of isooctane (8 ml/g) and stirred at room temperature. The product (0.36 g) was filtered off and washed with isooctane (4 ml/g) and was dried in room temperature. The product was confirmed with NMR and XRPD.

¹H-NMR (400 MHz, CDCl₃): 7.3-7.0 (7H, m), 6.7 (1H, d), 6.6 (1H, d), 4.6 (1H, s), 4.3 (1H, s), 3.7 (1H, m), 3.6 (1H, m), 3.5 (2H, m), 3.3 (1H, t), 3.1 (2H, m), 2.9 (1H, m), 2.7 (3H, m), 1.5 (2H, br m), 1.3 (9H, br s), 1.2 (6H, br s), 1.0 (3H, t), 0.8 (3H, m)

Properties

### Examples of properties of (1R,2S)-2-hydroxyindan-1-amine (2S)-2-ethoxy-3-(4-{2-[hexyl(2-phenylethyl)amino]-2-oxoethoxy}phenyl)propanoic acid

DSC showed an endotherm with an extrapolated onset temperature of 104° C. TGA showed a weight loss of 0% w/w between 24-75° C. DSC analysis repeated on purer sample may give a higher melting point. Crystals of (1R,2S)-2-hydroxyindan-1-amine (2S)-2-ethoxy-3-(4-{2-[hexyl(2-phenylethyl)amino]-2-oxoethoxy}phenyl)propanoic acid (obtained by way of the example above and/or by other ways) were analyzed by XRPD and the results are tabulated below and are shown in Figure A

[image]

### Figure A, XRPD pattern of (1R,2S)-2-hydroxyindan-1-amine (2S)-2-ethoxy-3-(4-{2-[hexyl(2-phenylethyl)amino]-2-oxoethoxy}phenyl)propanoic acid

| d-value | intensity |
|---|---|
| (Angstrom) | (rel) |
| 20.0 | S |
| 12.6 | M |
| 11.0 | Vs |
| 10.4 | M |
| 10.0 | M |
| 8.1 | m |
| 7.6 | m |
| 6.5 | s |
| 6.3 | w |
| 6.1 | m |
| 6.0 | m |
| 5.9 | s |
| 5.8 | s |
| 5.7 | s |
| 5.7 | m |
| 5.2 | m |
| 5.1 | s |
| 4.79 | m |
| 4.74 | m |
| 4.49 | m |
| 4.41 | s |
| 4.29 | m |
| 4.20 | m |
| 4.16 | w |
| 4.04 | s |
| 4.00 | w |
| 3.96 | m |
| 3.90 | s |
| 3.79 | m |
| 3.74 | m |
| 3.66 | s |
| 3.59 | w |
| 3.56 | w |
| 3.49 | w |
| 3.46 | w |
| 3.32 | w |
| 3.29 | w |
| 3.23 | w |
| 3.14 | w |
| 2.99 | w |
| 2.96 | w |
| 2.87 | m |
| 2.80 | w |
| 2.75 | w |
| 2.29 | w |

### Examples of L-arginine salt (2S)-2-ethoxy-3-(4-{2-[hexyl(2-phenylethyl)amino]-2-oxoethoxy}phenyl)propanoic acid

The (2S)-2-ethoxy3-(4-{2-[hexyl(2-phenylethyl)amino]-2-oxoethoxy}phenyl)propanoic acid (0.197 g) was dissolved in 95% ethanol and L-arginine (1 mole equiv) was added to the solution. The solution was stirred at ambient temperature, followed by evaporation to dryness and addition of isooctane (10 ml/g). The slurry was stirred at room temperature, and the product was filtered off and analysed by XRPD.

[image]

### Figure B: XRPD pattern for L-arginine salt (2S)-2-ethoxy-3-(4-{2-[hexyl(2-phenylethyl)amino]-2-oxoethoxy}phenyl)propanoic acid

### Examples of properties of tert-butylamine salt of (2S)-2-ethoxy-3-(4-{2-[hexyl(2-phenylethyl)amino]-2-oxoethoxy}phenyl)propanoic acid

DSC showed an endotherm with an extrapolated onset temperature of 107° C. TGA showed a weight loss of 12.7% w/w between 102-236° C. DSC analysis repeated on purer sample may give a higher melting point. Crystals of tert-butylamine salt of (2S)-2-ethoxy-3-(4-{2-[hexyl(2-phenylethyl)amino]-2-oxoethoxy}phenyl)propanoic acid (obtained by way of the example above and/or by other ways) were analyzed by XRPD and the results are tabulated below and are shown in Figure C

[image]

### Figure C, XRPD pattern of tert-butylamine salt of (2S)-2-ethoxy-3-(4-{2-[hexyl(2-phenylethyl)amino]-2-oxoethoxyphenyl}propanoic acid

| d-value | intensity |
|---|---|
| (Angstrom) | (rel) |
| 18.7 | vs |
| 11.5 | m |
| 10.4 | w |
| 8.7 | w |
| 8.1 | m |
| 7.3 | m |
| 6.9 | m |
| 6.7 | w |
| 6.3 | w |
| 5.9 | s |
| 5.8 | m |
| 5.5 | s |
| 5.2 | w |
| 5.1 | w |
| 5.00 | w |
| 4.86 | w |
| 4.71 | s |
| 4.44 | w |
| 4.24 | m |
| 4.08 | s |
| 4.02 | w |
| 3.77 | m |
| 3.74 | w |
| 3.67 | w |
| 3.53 | w |
| 3.14 | w |
| 3.06 | w |

Biological Activity

Compound A was tested in the assays described in WO 03/051821.

The compounds of the invention have an EC₅₀ of less than 0.5 μmol/l for PPARα and preferred compounds have an EC₅₀ of less than 0.05 μmol/l for PPARα. The compounds of the present invention are more potent with respect to PPARα than with respect to PPARγ. It is believed that this relationship is important with respect to the pharmacological activity of the compounds and to their therapeutic profile.

In addition the compounds of the present invention exhibit improved DMPK (Drug Metabolism and Pharmacokinetic) properties, for example they exhibit improved metabolic stability in vitro, and also exhibit favourable dose response curves in vivo. The compounds also have a promising toxicological profile.

### Further examples of salts of (2S)-2-ethoxy-3-(4-{2-[hexyl(2-phenylethyl)amino]-2-oxoethoxy}phenyl)propanoic acid

### Example 7

### Adamantylamine salt of (2S)-2-ethoxy-3-(4-{2-[hexyl(2-phenylethyl)amino]-2-oxoethoxy}phenyl)propanoic acid

(2S)-2-ethoxy-3-(4-{2-[hexyl(2-phenylethyl)amino]-2-oxoethoxy}phenyl)propanoic acid (0.32 g, 0.7 mmol) was dissolved in ethyl acetate (10 ml) in a round bottom flask. Adamantylamine (0.11 g, 0.7 mmol) was dissolved in a small portion of methylene chloride (2 ml) and the solution was added to the round bottom flask. The solvent was let to slowly evaporate at room temperature until one quarter of the solvent remained. The crystalls was isolated by filtration and dried under vacuum.

### Example 8

### A N-benzyl-2-phenylethanaminium salt of (2S)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (0.49 g) and N-benzyl-2-phenylethylamine (0.24 ml) were mixed in acetone (4 ml). Then isooctane (4 ml) was added and the slurry was stirred at room temperature overnight, and was then filtered off. The crystallinity for the product was confirmed with XRPD.

[image]

### Figure D: XRPD pattern for N-benzyl-2-phenylethanaminium salt of (2S)-2-ethoxy-3-(4-{2-[hexyl(2-phenylethyl)amino]-2-oxoethoxy}phenyl)propanoic acid

| d-value (Å) | Relative intensity |
|---|---|
| 13.3 | vs |
| 4.62 | s |
| 4.51 | m |
| 4.42 | s |
| 4.33 | s |
| 4.18 | w |
| 4.08 | w |
| 3.64 | w |
| 3.49 | m |
| 3.40 | w |
| 3.22 | m |

Definitions Used:

| % Relative Intensity* | Definition |
|---|---|
| 60-100 | vs (very strong) |
| 30-60 | s (strong) |
| 11-30 | m (medium) |
| 5-11 | w (weak) |
| <5 | vw (very weak) |

*The relative intensities are derived from diffractograms measured with variable slits.

### Example 9

### A N-benzyl-2-phenylethanaminium salt of (2S)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (0.98 g) and N-benzyl-2-phenylethylamine (0.52 ml) were mixed in IPA (1 ml). Then n-butyl acetate (4 ml) was added and seed was added. The slurry was stirred at room temperature for >72 hrs, and was then evaporated almost to dryness and filtered. The product was analysed by LC to confirm the assay.

### Example 10

### A N-benzyl-2-(benzylamino) ethanaminium salt of (2S)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (0.98 g) and N,N′-dibenzylethylenediamine (0.46 ml) were mixed in isopropanol (1 ml). Then n-butyl acetate (4 ml) was added and the slurry was stirred at room temperature >72 hrs, and was then filtered off. The product was analysed by LC to confirm the assay. The product was analysed with XRPD.

[image]

### Figur E: XRPD pattern for N-benzyl-2-(benzylamino) ethanaminium salt of (2S)-2-ethoxy-3-(4-{2-hexyl(2-phenylethyl)amino]-2-oxoethoxy}phenyl)propanoic acid

| d-value (Å) | Relative intensity |
|---|---|
| 16.2 | vs |
| 8.1 | m |
| 4.03 | vs |
| 3.66 | w |
| 3.30 | w |
| 3.22 | w |
| 2.30 | w |

Definitions Used:

| % Relative Intensity* | Definition |
|---|---|
| 60-100 | vs (very strong) |
| 30-60 | s (strong) |
| 11-30 | m (medium) |
| 5-11 | w (weak) |
| <5 | vw (very weak) |

*The relative intensities are derived from diffractograms measured with variable slits.

### Example 11

### A N-benzyl-2-(benzylamino) ethanaminium salt of (2s)-2-ethoxy-3-(4-{2-[hexyl(2-phenylethyl)amino]-2-oxoethoxy}phenyl)propanoic acid

(2S)-2-Ethoxy-3-(4-{2-[hexyl(2-phenylethyl)amino]-2-oxoethoxy}phenyl)propanoic acid (0.98 g) and N,N′-dibenzylethylenediamine (0.52 ml) were mixed in IPA (1 ml). Then n-butyl acetate (4 ml) was added and seed was added. The slurry was stirred at room temperature for >72 hrs, and was then evaporated almost to dryness and filtered. The product was analysed by LC to confirm the assay.

1. A compound selected from one or more of the following:
a (1R,2S)-2-hydroxyindan-1-amine salt of (2S)-2-ethoxy-3-(4-{2-[hexyl(2-phenylethyl)amino]-2-oxoethoxy}phenyl)propanoic acid;
an L-arginine salt of (2S)-2-ethoxy-3-(4-{2-[hexyl(2-phenylethyl)amino]-2-oxoethoxy}phenyl)propanoic acid;
a tert-butylamine salt of (2S)-2-ethoxy-3-(4-{2-[hexyl(2-phenylethyl)amino]-2-oxoethoxy}phenyl)propanoic acid;
a choline salt of (2S)-2-ethoxy-3-(4-{2-[hexyl(2-phenylethyl)amino]-2-oxoethoxy}phenyl)propanoic acid;
an adamantylamine salt of (2S)-2-ethoxy-3-(4-{2-[hexyl(2-phenylethyl)amino]-2-oxoethoxy}phenyl)propanoic acid;
a N-benzyl-2-phenylethanaminium salt of (2S)-2-ethoxy-3-(4-{2-[hexyl(2-phenylethyl)amino]-2-oxoethoxy}phenyl)propanoic acid;
a N-benzyl-2-(benzylamino) ethanaminium salt of (2S)-2-ethoxy-3-(4-{2-[hexyl(2-phenylethyl)amino]-2-oxoethoxy}phenyl)propanoic acid; or
a tris(hydroxymethyl)methylamine salt of (2S)-2-ethoxy-3-(4-{2-[hexyl(2-phenylethyl)amino]-2-oxoethoxy}phenyl)propanoic acid.

2. A compound according to claim 1 wherein the compound is selected from one or more of the following:
a (1R,2S)-2-hydroxyindan-1-amine salt of (2S)-2-ethoxy-3-(4-{2-[hexyl(2-phenylethyl)amino]-2-oxoethoxy}phenyl)propanoic acid;
an L-arginine salt of (2S)-2-ethoxy-3-(4-{2-[hexyl(2-phenylethyl)amino]-2-oxoethoxy}phenyl)propanoic acid;
a tert-butylamine salt of (2S)-2-ethoxy-3-(4-{2-[hexyl(2-phenylethyl)amino]-2-oxoethoxy}phenyl)propanoic acid;
a choline salt of (2S)-2-ethoxy-3-(4-{2-[hexyl(2-phenylethyl)amino]-2-oxoethoxy}phenyl)propanoic acid; or
a tris(hydroxymethyl)methylamine salt of (2S)-2-ethoxy-3-(4-{2-[hexyl(2-phenylethyl)amino]-2-oxoethoxy}phenyl)propanoic acid.

3. A compound according to claim 1 wherein the compound is selected from:
an adamantylamine salt of (2S)-2-ethoxy-3-(4-{2-[hexyl(2-phenylethyl)amino]-2-oxoethoxy}phenyl)propanoic acid;
a N-benzyl-2-phenylethanaminium salt of (2S)-2-ethoxy-3-(4-{2-[hexyl(2-phenylethyl)amino]-2-oxoethoxy}phenyl)propanoic acid; or
a N-benzyl-2-(benzylamino) ethanaminium salt of (2S)-2-ethoxy-3-(4-{2-[hexyl(2-phenylethyl)amino]-2-oxoethoxy}phenyl)propanoic acid.

4. A compound selected from:
a (1R,2S)-2-hydroxyindan-1-amine salt of (2S)-2-ethoxy-3-(4-{2-[hexyl(2-phenylethyl)amino]-2-oxoethoxy}phenyl)propanoic acid;
an L-arginine salt of (2S)-2-ethoxy-3-(4-{2-[hexyl(2-phenylethyl)amino]-2-oxoethoxy}phenyl)propanoic acid; or
a tert-butyl amine salt of (2S)-2-ethoxy-3-(4-{2-[hexyl(2-phenylethyl)amino]-2-oxoethoxy}phenyl)propanoic acid.

5. A salt as claimed in any one of claims 1 to 4 which may be a solvate, a hydrate, a mixed solvate/hydrate, an ansolvate or an anhydrate.

6. A salt as claimed in any one of claims 1 to 4 in crystalline or partially crystalline form.

7. A salt as claimed in claim 5 in crystalline or partially crystalline form.
